(19) United States
(12) Patent Application Publication
(10) Pub. No.: US 2015/0226815 A1
(43) Pub. Date: Aug. 13, 2015

(54) AUTOMATIC BOLUS DETECTION

(71) Applicants: Kai Tobias Block, New York, NY (US); Robert Grimm, Nuernberg (DE); Marcel Dominik Nickel, Erlangen (DE)

(72) Inventors: Kai Tobias Block, New York, NY (US); Robert Grimm, Nuernberg (DE); Marcel Dominik Nickel, Erlangen (DE)

(21) Appl. No.: 14/178,741

(22) Filed: Feb. 12, 2014

(51) Int. Cl. G01R 33/28; G01R 33/48; A61B 5/055

(52) U.S. Cl. CPC G01R 33/281; A61B 5/055; G01R 33/4818

(57) ABSTRACT

In a method for automatically detecting contrast enhancement at predetermined phases as a contrast agent bolus perfuses a target tissue volume in a patient, a continuous acquisition MRI imaging system is provided for obtaining dynamic contrast enhanced MRI data for use in creating images. The contrast agent bolus is injected into a blood stream of the patient which passes through the target volume. With the imaging system, a center of a k-space of the target volume is repeatedly sampled to obtain k-space data. A bolus time curve signal is automatically extracted from the k-space data which indicates a course of bolus contrast enhancement which is used to automatically pick time frames at the predetermined phases of the perfusion which are then used to identify corresponding key images to be obtained at the time frames.

### BACKGROUND

In contrast enhanced MRI (also known as Dynamic Contrast Enhanced (DCE)-MRI (contrast agent perfusion imaging) reconstruction of 3D volumes at high temporal resolution has become technically feasible. More particularly, in clinical 3D Dynamic Contrast-Enhanced MRI (DCE-MRI) of the abdomen, multiple phases of perfusion (pre-contrast, arterial, venous, and delayed phases) are captured subsequently in breath-hold scans. See Michaely et al., CAIPIRINHA-Dixon-TWIST (CDT)-Volume-Interpolated Breath-Hold Examination (VIBE), Investigative Radiology 48(8), 2013. Conventionally, predefined delays have been used to acquire an image at the estimated time of specific phases of perfusion.

As shown in prior art FIG. 1, two different phases of contrast enhancement in liver imaging are illustrated generally at 10. The initial native phase is illustrated at 11 followed by injection of the image contrast bolus at 12. Thereafter an initial bolus detection at 13 occurs followed by the contrast enhancement phases arterial at 14, venous at 15, and delay (late) at 16. Predefined time delays 17 and 18 are shown for the arterial phase and the venous phase, respectively. See, Martin et al., Challenges and Clinical Value of Automated and Patient-Specific Dynamically Timed CE Liver MRI Examination. MAGNETOM Flash 3: 40-45, 2009.

Novel imaging and reconstruction techniques such as Golden-Angle Radial Sparse Parallel (GRASP) (see Feng et al. #0081, ISMRM 2012) promise DCE-MRI at a temporal resolution of only a few seconds from a single, continuous image acquisition. This reduces the requirements on bolus timing accuracy and can thereby significantly simplify the imaging workflow. As an example, the course of contrast enhancement over a few minutes can be captured by 100 temporal steps. For most clinical diagnoses, however, only a fraction of these images is actually relevant: for instance the “pre-contrast” (native), “arterial”, “portal venous”, “venous”, and “late” phases of contrast enhancement in liver imaging.

The large amount of data is difficult to handle in terms of visualization, interpretation and storage.

A k-space of a Golden-Angle Radial Sparse Parallel (GRASP) MRI imaging method is schematically illustrated at 19 in prior art FIG. 2 wherein the GRASP method has the following characteristics:
continuous data acquisition with stack-of-stars trajectory
compressed sensing reconstruction exploits temporal sparsity
flexible temporal resolution (e.g., 3 s). For this, timing is not critical, and a high number of images can be obtained. See Feng et al. #1117, Proc. ISMRM 2012 and Block et al. #3809, Proc. ISMRM 2013.

FIG. 3 illustrates generally at 20 the various contrast enhancement phases in liver imaging known in the prior art where a plurality of images are obtained using the GRASP technique. Here a plurality of GRASP images 21a-21h are illustrated where some of these images correspond to the native, arterial, venous, and delayed phases. Bolus injection is shown at 22 and bolus detection 23 occurs in image 21b.

However, the GRASP technique also has two disadvantages that have not been solved so far. First, it cannot be combined with conventional bolus detection techniques (see Shama et al., JMRI 33, p. 110, 2011 and Hussain et al. Radiology 226, 2003) to monitor the contrast agent (CA) bolus (contrast dose). Because the reconstruction is computationally so intensive that dynamic images are computed with significant delay, no direct visual feedback is available after the scan. Second, the resulting 4D images that can comprise more than 100 time-steps (see Kim et al. #1468, ISMRM 2012) impose a significant amount of data that cannot be adequately visualized or analyzed with most clinical imaging software. Identifying the few critical phases of perfusion in the time series requires manual interaction from the radiologist or carefully tuned, application-specific segmentation algorithms (see Chen et al. LNCS 5241, p. 594, 2008).

### SUMMARY

It is an object to provide for automatic detection of contrast enhancement at predetermined phases.

In a method for automatically detecting contrast enhancement at predetermined phases as a contrast agent bolus perfuses a target tissue volume in a patient, a continuous acquisition MRI imaging system is provided for obtaining dynamic contrast enhanced MRI data for use in creating images. The contrast agent bolus is injected into a blood stream of the patient which passes through the target volume. With the imaging system, a center of a k-space of the target volume is repeatedly sampled to obtain k-space data. A bolus time curve signal is automatically extracted from the k-space data which indicates a course of bolus contrast enhancement which is used to automatically pick time frames at the predetermined phases of the perfusion which are then used to identify corresponding key images to be obtained at the time frames.

### BRIEF DESCRIPTION OF THE DRAWINGS

FIG. 1 illustrates two different phases of contrast enhancement in liver imaging;

FIG. 2 illustrates a k-space Golden-Angle Radial Sparse Parallel (GRASP) MRI imaging;

FIG. 3 illustrates various contrast enhancement phases in liver imaging known in the prior art utilizing the GRASP technique;

FIG. 4 illustrates a k-space utilized for bolus signal detection;

FIG. 5 illustrates a bolus signal curve which is obtained;

FIG. 6 illustrates a bolus analysis;

FIG. 7 illustrates a bolus signal with a solid line and a fitted model with a dash line;

FIG. 8 shows automatically selected free-contrast, arterial, and venous phase images; and

FIG. 9 shows a computer system for implementing the automatic bolus detection of the exemplary embodiments.

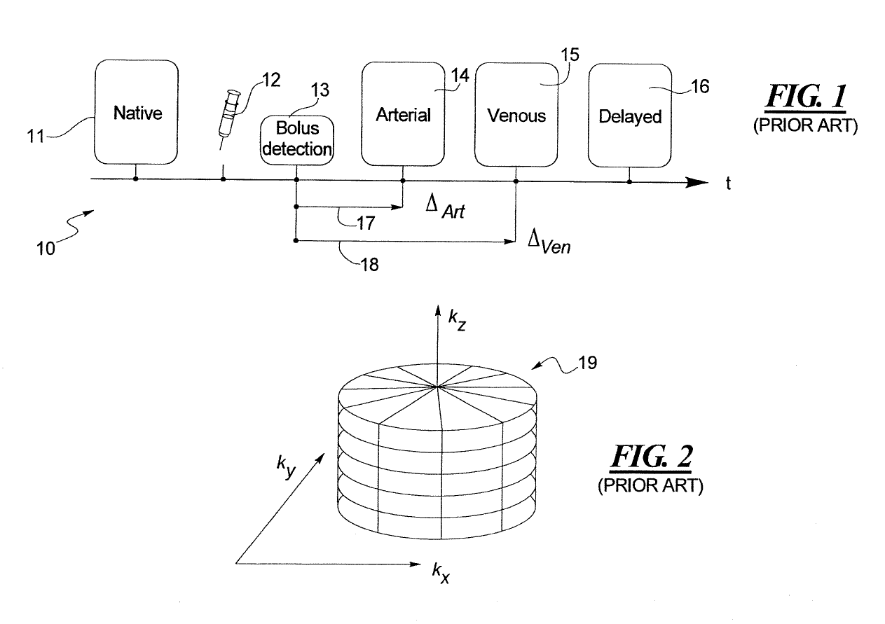

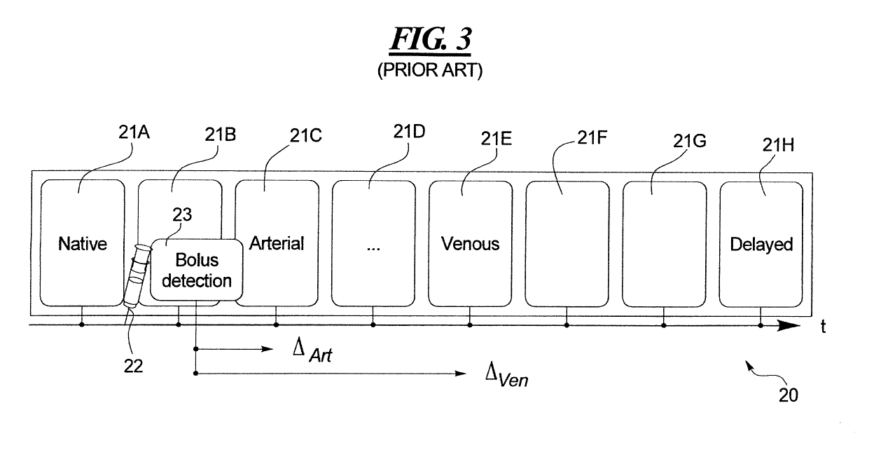

### DESCRIPTION OF EXEMPLARY EMBODIMENTS

For purposes of promoting an understanding of the principles of the invention, reference will now be made to the preferred exemplary embodiments/best mode illustrated in the drawings and specific language will be used to describe the same. It will nevertheless be understood that no limitation of the scope of the invention is thereby intended, and such alterations and further modifications in the illustrated embodiments and such further applications of the principles of the invention as illustrated as would normally occur to one skilled in the art to which the invention relates are included herein.

With the disclosed exemplary embodiment method, information about the course of contrast enhancement that is available from the sampled data is taken advantage of.

As shown in FIG. 9 discussed in more detail hereafter, a computer system 27 is provided for performing an automatic bolus analysis (detection) of k-space data output from a continuous acquisition DCE-MRI Golden-Angle Radial Sparse Parallel (GRASP) stack-of-stars Gradient Echo (GRE) Imaging system 35. A parameter-free method is provided to automatically extract a bolus time curve from raw k-space data acquired with a radial stack-of-stars GRE sequence. The computer system 27 is used to display on a computer display 36 statistics about the contrast agent (CA) bolus right after the scan, as well as to automatically pick time frames at important stages of perfusion. Because the approach is k-space-based, the temporal accuracy is not limited by the reconstructed images.

With an according k-space trajectory, for example a “radial stack of stars” or “radial phase encoding”, a k-space center kx=ky=kz=0 is sampled repeatedly. It reflects the global course of contrast enhancement in the target volume and thus allows to automatically detect arrival of a contrast agent bolus.

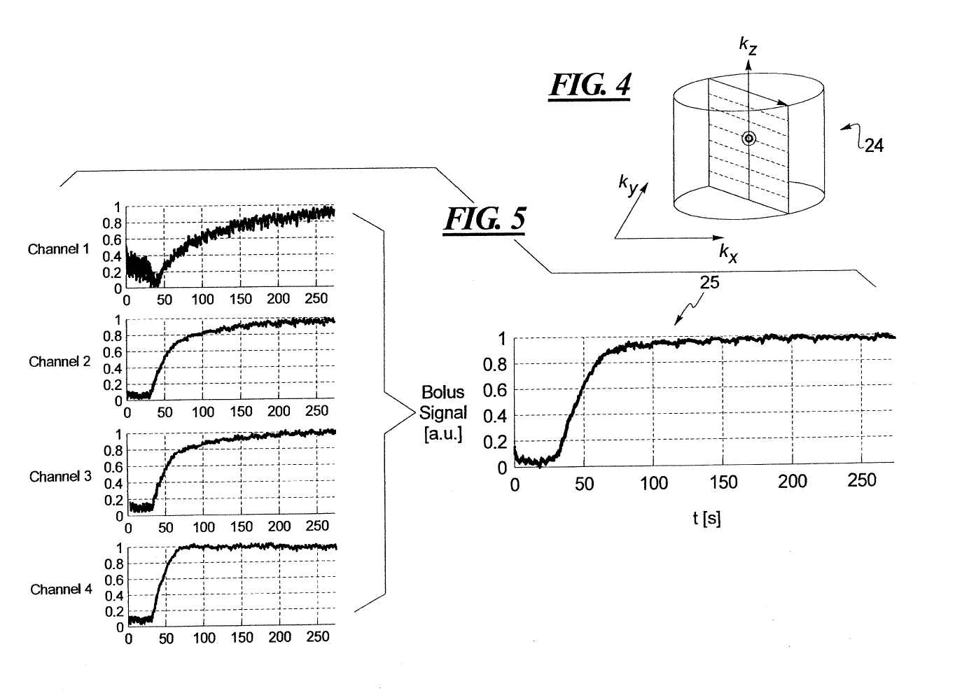

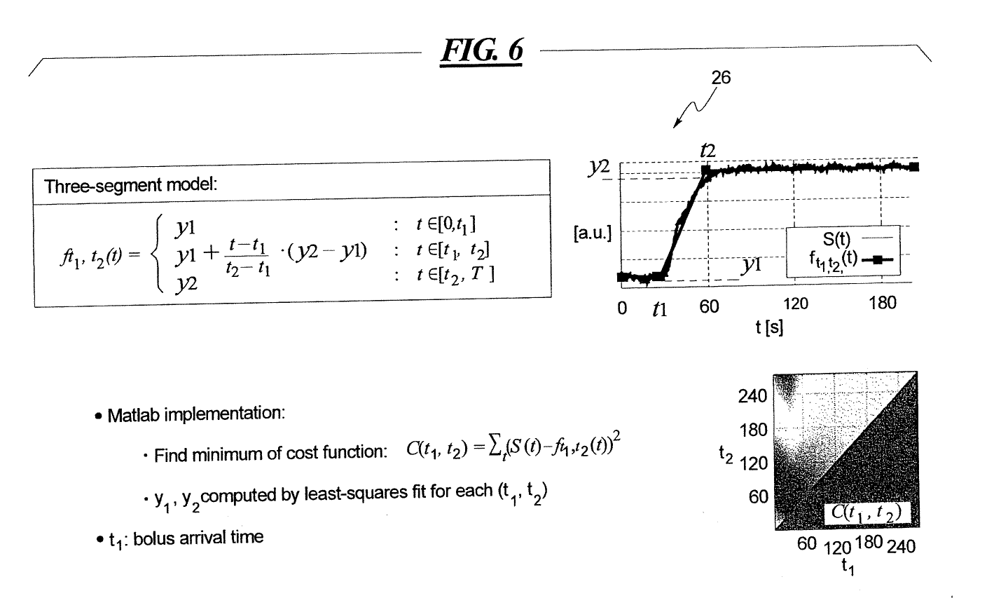

Bolus signal detection utilizing the k-space 24 in FIG. 4 is shown in FIG. 5 illustrating a bolus signal curve 25. The detection is similar to self-gating wherein one extracts the k-space center kₓ=k_{y}=k_{z}=0, and Principal Component Analysis (PCA) compression is applied to reduce multi-channel data to a 1D signal. See Lin et al. Respiratory Motion-Compensated DCE-MRI of Chest and Abdominal Lesions. MRM 60:1135-1146, 2008; and Grimm et al. Optimal Channel Selection for Respiratory Self-Gating Signals. #3749, ISMRM 2013.

Optionally, a 1D Fourier transform along a slice encoding dimension is applied, which allows a restriction of the volume of interest for the bolus signal to certain slices, e.g. containing a heart.

The observed enhancement scheme allows a deduction of information about physiological phases of perfusion. Relevant volumes of the perfusion series are also determined e.g. by using predefined delays after characteristic features of the bolus curve, such as a beginning of the enhancement.

Another application is the use of the extracted signal as prior knowledge for actual image reconstruction. For instance, it is used to guide a temporal filter to preserve the temporal resolution during the most critical phases of perfusion.

Compared to conventional, image-based methods, this new method has the advantage that the bolus signal is sampled every time a readout crosses the k-space center, thus allowing for a potentially much higher temporal update rate. Moreover, no images actually have to be reconstructed, making it computationally more efficient.

A more detailed explanation of the method will now be provided.

The method is related to respiratory self-gating techniques that have been proposed for MRI with radial k-space trajectories (see Lin et al. MRM 60, p. 1135, 2008 and Grimm et al. #0598, ISMRM 2012). The course of contrast enhancement causes an increase in the total transverse magnetization, which is reflected in the magnitude of the central samples of every radial spoke in the k-space center partition (k_{z}=0). With this technique, a 1D signal can be extracted for every acquired channel. PCA compression (see Buehrer et al. MRM 57, p. 1131, 2007) is then applied to reduce the multi-channel data to a single 1D signal.

FIG. 6 illustrates generally at 26 the bolus analysis used in the exemplary embodiment method. The method illustrated in FIG. 6 may be further explained as follows.

The typical time course of enhancement in a volume is a constant section before contrast agent (CA) injection, followed by a rapid signal increase at bolus arrival and a slow wash-out. These three phases are modeled using a constant, a linear, and another constant line segment. This model requires only two degrees of freedom, referred to as x₁ and x₂ in the following. The pre-contrast segment ends at time point x₁ while the washout begins at x₂. The model is fitted by exhaustive search using the following cost function:

$${f ( {x}_{1} , {x}_{2} ) = \begin{matrix}{\sum }_{i = 1}^{{x}_{1}} {( {B}_{i} - {y}_{1} ( {x}_{1} ) )}^{2} + {\sum }_{i = {x}_{1} + 1}^{{x}_{2} - 1} {B}_{i} - \\ \frac{{y}_{2} ( {x}_{2} ) - {y}_{1} ( {x}_{1} )}{{x}_{2} - {x}_{1}} ( i - {x}_{1} ) {y}_{1} ( {x}_{1} )\end{matrix} )}^{2} + {\sum }_{i = {x}_{2}}^{N} {( {B}_{i} - {y}_{2} ( {x}_{2} ) )}^{2} ) ,$$

where Bᵢ is the i-th sample in the enhancement signal B of length N, and y₁(x₁) and y₂(x₂) are the values obtained by least-squares fitting of a constant line segment to the first x₁ (or last N−x₂+1) samples of the enhancement signal.

The ratio of the distance between the constant segments to the standard deviation of the signal during the whole acquisition, (y₂−y₁)/std(B), can be used as a simple indicator of actual contrast enhancement. The onset time x₁ and the plateau time x₂ provide additional checks whether the bolus arrival was truly captured by the acquisition. After image reconstruction, the critical phases of perfusion can be found by using population-based estimates for the respective delays from the detected bolus time x₁.

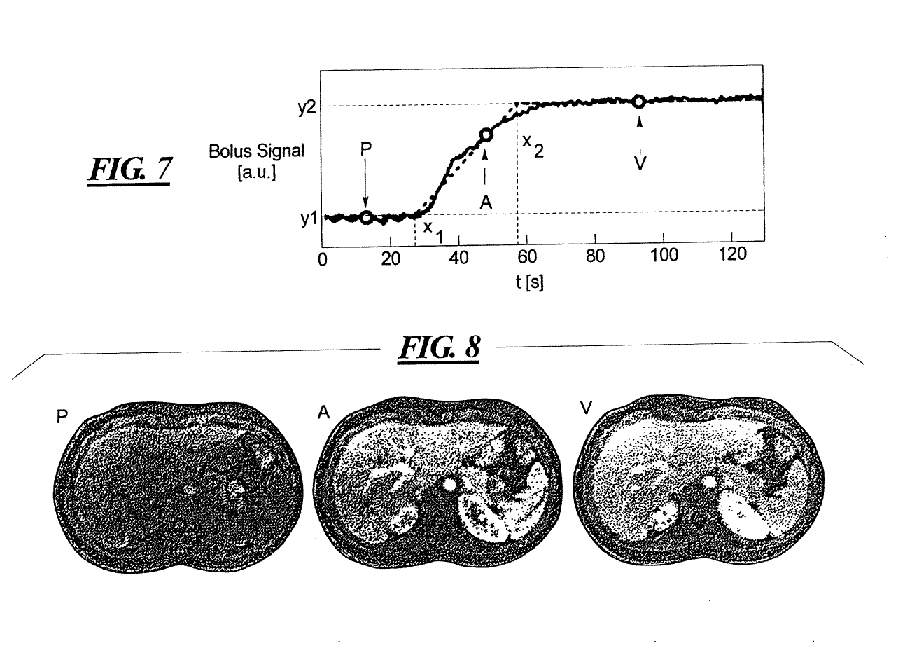

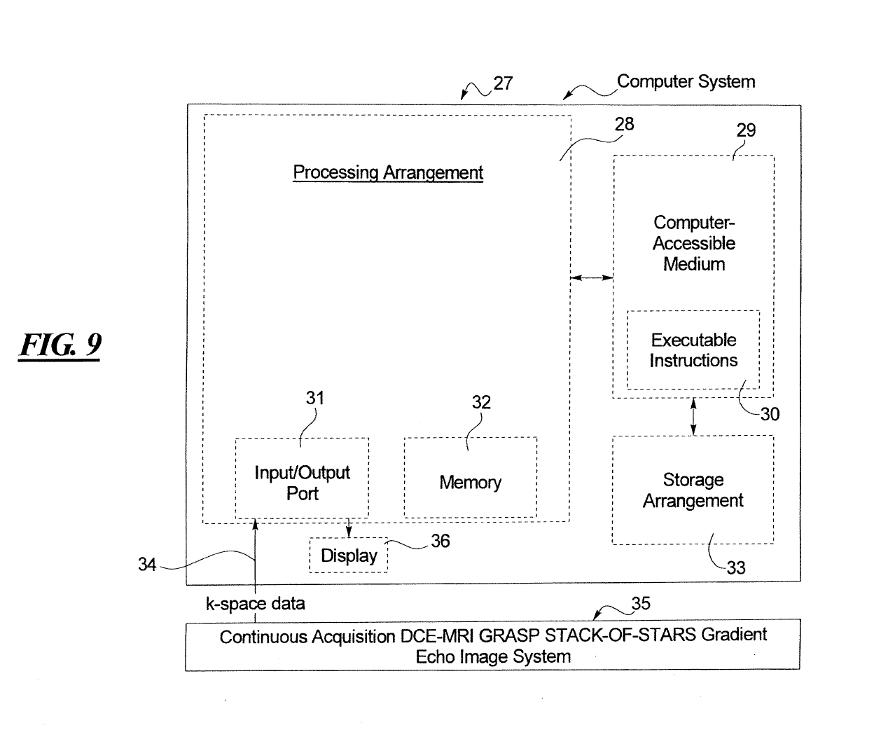

FIG. 7 shows the Bolus signal with a line and the fitted model with a dashed line. FIG. 8 shows automatically selected pre-contrast (P), arterial (A), and venous (V) phase images.

The correctness of the images is confirmed visually, as shown in FIG. 8: no enhancement in the pre-contrast image P, maximal enhancement of the portal vein but no enhancement of hepatic veins in the arterial phase A, and enhancement of all vessels in the venous phase V.

The disclosed method allows fully automatic extraction of a signal characterizing the course of contrast enhancement in golden-angle radial (GRASP) DCE-MRI acquisitions. Fitting a three-segment model is used to precisely detect the bolus arrival, making it possible to immediately recognize bolus cases where the bolus administration failed. Using population-based estimates for the delay of the arterial and venous phases of perfusion, the detected bolus onset is used to automatically extract the clinically relevant key images from a dynamic time series.

In summary, in the disclosed method to find key images in abdominal (such as liver) DCE-MRI, the following occurs:
automatic detection of bolus in k-space center
extraction of images at empirical timing delays
based on data rather than images, a higher temporal resolution occurs which is not subject to reconstruction artifacts.

FIG. 9 illustrates the previously described computer system 27 receiving the k-space data on data line 34 from the continuous acquisition MRI imaging system 35 for implementing the automatic bolus detection of the exemplary embodiments.

As shown in FIG. 9, e.g., a computer-accessible medium 120 (e.g., as described herein, a storage device such as a hard disk, floppy disk, memory stick, CD-ROM, RAM, ROM, etc., or a collection thereof) is provided (e.g., in communication with the processing arrangement 28). The computer-accessible medium 29 is a non-transitory computer-accessible medium. The computer-accessible medium 29 can contain executable instructions 30 thereon. In addition or alternatively, a storage arrangement 33 is provided separately from the computer-accessible medium 29, which provides the instructions to the processing arrangement 28 so as to configure the processing arrangement to execute certain exemplary procedures, processes and methods, as described herein, for example.

Computer system 27 also includes a display or output device 36, an input device such as a key-board, mouse, touch screen or other input device, and may be connected to additional systems via a logical network. Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Although preferred exemplary embodiments are shown and described in detail in the drawings and in the preceding specification, they should be viewed as purely exemplary and not as limiting the invention. It is noted that only preferred exemplary embodiments are shown and described, and all variations and modifications that presently or in the future lie within the protective scope of the invention should be protected.

We claim as our invention:

1. A method for automatically detecting contrast enhancement at predetermined phases as a contrast agent bolus perfuses a target tissue volume in a patient, comprising the steps of:
providing a continuous acquisition MRI imaging system for obtaining dynamic contrast enhanced MRI data for use in creating images;
injecting the contrast agent bolus into a blood stream of the patient which passes through said target volume;
with the imaging system repeatedly sampling a center of a k-space of said target volume to obtain k-space data; and
automatically extracting from said k-space data a bolus time curve signal indicating a course of bolus contrast enhancement which is used to automatically pick time frames at said predetermined phases of said perfusion which are then used to identify corresponding key images to be obtained at said time frames.

2. The method of claim 1 wherein the continuous acquisition MRI imaging system obtains the dynamic contrast enhanced MRI data by radial acquisition and which is output as said k-space data.

3. The method of claim 2 wherein the continuous acquisition MRI imaging system for obtaining the dynamic contrast enhanced MRI data by radial acquisition comprises a golden-angle radial sparse parallel stack-of-stars gradient echo system for obtaining the dynamic contrast enhanced MRI data which is output as said k-space data.

4. The method of claim 1 wherein the contrast agent bolus perfuses in both blood vessels and tissue of the target tissue volume.

5. The method of claim 1 wherein said predetermined phases are pre-contrast, arterial, and venous phase, and wherein said key images are obtained for those phases.

6. The method of claim 1 wherein no direct visual feedback of images derived from said k-space data are employed prior to identification of said key images.

7. The method of claim 1 wherein said bolus time curve signal is derived by sampling any time a read out crosses the center of the k-space.

8. The method of claim 1 wherein the extracted bolus time curve signal is used as prior knowledge for actual image reconstruction prior to obtaining any images from said k-space data.

9. The method of claim 1 wherein only the key images are obtained corresponding to said predetermined phases of said perfusion.

10. The method of claim 1 wherein a three-segment model is used which is compared to said bolus time curve signal to precisely detect bolus arrival at locations within said target volume.

11. The method of claim 1 wherein said k-space data for extracting said bolus time curve signal is spatially resolved along one or multiple directions for obtaining localized contrast-enhancement phases.

12. A system for automatically detecting contrast enhancement at predetermined phases as a contrast agent bolus perfuses a target tissue volume in a patient, comprising:
a continuous acquisition MRI imaging system for obtaining dynamic contrast enhanced MRI data for use in creating images, said system, after injection of the contrast agent bolus into a blood stream of the patient which passes through said target volume, repeatedly sampling a center of a k-space of said target volume to obtain k-space data; and
a computer system comprising a processing arrangement with a memory, an input/output port, a display, and a computer-accessible medium, said input/output port being connected to receive said k-space data and being connected to output to said display, and said computer-accessible medium containing instructions which when executed by said processing arrangement automatically extracting from said k-space data a bolus time curve signal indicating a course of bolus contrast enhancement which is used to automatically pick time frames at said predetermined phases of said perfusion which are then used to identify corresponding key images to be obtained at said time frames, the key images being identified on said display.

13. The system of claim 12 wherein said MRI imaging system comprises a radial acquisition system.

14. The system of claim 13 wherein said MRI radial acquisition imaging system comprises a golden-angle radial sparse parallel stack-of-stars gradient echo system.

15. The system of claim 12 wherein said predetermined phases are pre-contrast, arterial, venous phase, and wherein said key images are obtained for those phases.

16. A non-transitory computer-accessible medium for use in automatically detecting contrast enhancement at predetermined phases as a contrast agent bolus perfuses a target tissue volume in a patient, and wherein a continuous acquisition MRI imaging system is provided for obtaining dynamic contrast enhanced MRI data for use in creating images, said system repeatedly sampling a center of a k-space of said target volume to obtain k-space data and wherein a contrast agent bolus is injected into a blood stream of the patient which passes through said target volume, said medium containing a program which when executed by a computer system automatically extracts from the k-space data a bolus time curve signal indicating a course of bolus contrast enhancement which is used to automatically pick time frames at said determined phases of said perfusion which are then used to identify corresponding key images to be obtained at said time frames, said identified key images being output by said computer system.

17. The medium of claim 16 wherein the continuous acquisition MRI imaging system comprises a radial acquisition system.

18. The medium of claim 17 wherein the MRI radial acquisition imaging system comprises a golden-angle radial sparse parallel stack-of-stars gradient echo system.

19. The medium of claim 16 wherein said predetermined phases are pre-contrast, arterial, and venous phase, and wherein said key images are obtained for those phases.
